(19) United States
(12) Patent Application Publication
(10) Pub. No.: US 2023/0320973 A1
(43) Pub. Date: Oct. 12, 2023

(54) ULTRAVIOLET-ABSORPTIVE NANOPARTICLES AND MICROPARTICLES FOR INTRADERMAL USE

(71) Applicant: THE REGENTS OF THE UNIVERSITY OF COLORADO, a body corporate, Denver, CO (US)

(72) Inventors: Carson J. Bruns, Boulder, CO (US); Jesse Butterfield, Boulder, CO (US)

(21) Appl. No.: 18/042,509

(22) PCT Filed: Aug. 27, 2021
(86) PCT No.: PCT/US21/47941

§ 371 Date: Feb. 22, 2023

Related U.S. Application Data
(60) Provisional application No. 63/071,782, filed on Aug. 28, 2020.

(51) Int. Cl. A61K 8/85; A61K 8/49; A61Q 17/04; A61K 8/29; A61K 8/27; A61K 8/19; A61K 8/81; A61K 8/02

(52) U.S. Cl. CPC A61K 8/85; A61K 8/4926; A61Q 17/04; A61K 8/29; A61K 8/27; A61K 8/4966; A61K 8/19; A61K 8/8147; A61K 8/0279; A61K 2800/91; A61K 2800/654; A61K 2800/43; A61K 2800/651

(57) ABSTRACT

Biocompatible UV-absorbing nanoparticles or microparticles that can be embedded in the skin using techniques such as those used to create a tattoo with tattoo ink. The “tattoo” using the biocompatible UV-absorbing nanoparticles or microparticles provides skin protection against sunburn, photoaging, and skin cancers in a permanent or semi-permanent way, but remain clear in the visible light spectrum, or matched closely to the user's specific skin tone. These particles can be solid uniform UV-absorbers, microencapsulated UV-absorbers, or UV-absorbing material embedded in or coated on solid materials. Long-term sun protection from an invisible (does not change the color of the skin) material, embedded in the skin (dermis layer).

### CROSS-REFERENCE TO RELATED APPLICATIONS

This application is the National Phase under 35 U.S.C. § 371 of International Application No. PCT/US21/47941, filed Aug. 27, 2021, which claim the benefit of priority to U.S. Provisional Application No. 63/071,782, filed Aug. 28, 2020. The entire contents of these patent applications are hereby incorporated by reference herein.

### FIELD OF INVENTION

This invention relates to compositions and methods for producing biocompatible UV-absorbing microparticles.

### BACKGROUND OF THE INVENTION

Ultraviolet (UV) radiation is the main risk factor for skin cancer (both melanoma and non-melanoma), which is the most common malignancy (more common than all other cancers combined) in the United States and other predominantly light-skinned populations worldwide. [Diepgen, T. L.; Mahler, V. The epidemiology of skin cancer. Br. J Derm. 2002, 146, 1-6.] Most of the UV rays transmitting through the earth's atmosphere are UVA (320-400 nm wavelength), while a small amount of UVB rays (280-320 nm wavelength) also reach the earth's surface. Exposure to both UVA and UVB leads to cumulative skin damage over time, increasing skin cancer risk and aging rates. [Taylor, C. R.; Stern, R. S.; Leyden, J. J.; Gilchrest, B. A Photoaging/Photodamage and Photoprotection. J Am. Acad Dermatol. 1990, 22, 1-15.] UVB is the primary cause of sunburn and the main risk factor for melanoma (one of the least common, but most lethal skin cancers), while the more deeply penetrating UVA rays are associated with skin aging and increase the risk of the most common keratinocyte carcinomas. [Albert, M. R.; Weinstock, M. A Keratinocyte Carcinoma. CA Cancer J Clin. 2003, 53, 292-302.]

For areas of the skin that are not protected by clothing in sunlight, the recommended UV protection strategy is the use of a broad-spectrum topical sunscreen with a sun protection factor (SPF) of 15 or higher. [Koh, H. K.; Geller, AC.; Miller, D. R.; Grossbart, T. A; Lew, R. A. Prevention and Early Detection Strategies for Melanoma and Skin Cancer: Current Status. Arch. Dermatol. 1996, 132, 436-443.] The SPF rating applies only to UVB light; an SPF N sunscreen is rated to reduce incident UVB irradiance to a fraction of 1/N. Unfortunately, the sixteen different ingredients approved by the U.S. Food and Drug Administration (FDA) for over-the-counter sunscreens offer variable protection from UVA radiation, and recent rules proposed by the FDA suggest there is insufficient data to prove their safety in most cases. [US Food and Drug Administration. Sunscreen Drug Products for Over-the-Counter Human Use: Proposed Rule. Federal Register 2019, 84, 6204-6275.] It was recently found that the FDA-approved organic sunscreen ingredients can migrate into the bloodstream and exceed the 0.5 ng/mL concentrations threshold set by the FDA to waive nonclinical toxicology studies. [Matta, M. K. et al., Effect of Sunscreen Application Under Maximal Use Conditions on Plasma Concentration of Sunscreen Active Ingredients: A Randomized Clinical Trial. JAMA 2019, 21, 2082-2091.] Furthermore, the invisibility and comfort of sunscreen make it difficult to assess one's own coverage and know when to re-apply, and <30% of US adults use sunscreen appropriately. [Holman, D. M. et al., Patterns of Sunscreen Use on the Face and Other Exposed Skin Among US Adults. J Am. Acad Dermatol. 2015, 73, 83-92.El.] The risks, difficulty and inconvenience of proper application to the skin, and low prevalence of sunscreen use motivate the innovation of new UV-protective strategies for exposed skin.

### SUMMARY OF THE INVENTION

The present invention provides biocompatible UV-absorbing nanoparticles or microparticles that can be embedded in the skin using techniques such as those used to create a tattoo with tattoo ink. The “tattoo” using the biocompatible UV-absorbing nanoparticles or microparticles can provide skin protection against sunburn, photoaging, and skin cancers in a permanent or semi-permanent way, but remains clear in the visible light spectrum, or can be matched closely to the user's specific skin tone. These particles can be, for example, solid uniform UV-absorbers, microencapsulated UV-absorbers, or UV-absorbent material embedded in solid materials. Long-term sun protection from an invisible (does not significantly change the color of the skin) material, embedded in the skin (dermis layer).

An exemplary biocompatible UV-absorbing microparticle is poly(methyl methacrylate) (PMMA) in combination with a commercially-available UV absorber (for example, sunscreens). Some examples of materials that could be used as UV absorbers and photostabilizers include 2-hydroxybenzophenone, hydroxyphenyl-s-triazine, 2-(2-hydroxyphenyl)benzotriazole, oxalanilide, Aminobenzoic acid, Avobenzone, Cinoxate, Dioxybenzone, Homosalate, Meradimate, Octocrylene, Octinoxate, Octisalate, Oxybenzone, Padimate 0, Ensulizole, Sulisobenzone, Titanium dioxide, Trolamine salicylate, Zinc oxide (including derivatives of the aforementioned compounds). The UV absorber can be combined with a polymer material such as PMMA, polylactic acid (PLA), poly(lactic-co-glycolic acid) (PLGA), poly(dimethylsiloxane) (PDMS), polyethylene glycol (PEG), Melamine-formaldehyde, Methacrylamide chitosan, and many others.

In a first aspect the present invention provides an ultraviolet (UV) light-absorbing particle comprising poly(methyl methacrylate) (PMMA) in combination with a UV absorber. In an advantageous embodiment the UV absorber is a commercially-available UV absorber. In a particularly advantageous embodiment of the first aspect the commercially-available UV absorber is 2-hydroxybenzophenone, hydroxyphenyl-s-triazine, and 2-(2-hydroxyphenyl)benzotriazole, oxalanilide, Aminobenzoic acid, Avobenzone, Cinoxate, Dioxybenzone, Homosalate, Meradimate, Octocrylene, Octinoxate, Octisalate, Oxybenzone, Padimate 0, Ensulizole, Sulisobenzone, Titanium dioxide, Trolamine salicylate, Zinc oxide or derivatives and/or combinations thereof. In certain embodiments the ultraviolet light-absorbing particle according to the first aspect is a core-shell particle or nano/microcapsule having a core comprising a UV absorber within a shell or capsule comprising PMMA. In further embodiments the ultraviolet light-absorbing particle according to the first aspect can be a UV absorber that is randomly dispersed in a PMMA matrix

In a second aspect the present invention provides a second ultraviolet light-absorbing particle. The ultraviolet light-absorbing particle according to the second aspect can include a biocompatible polymer in combination with a commercially-available UV absorber. In certain embodiments of the second aspect the UV absorber can be a UV absorber belonging to the family of hydroxyphenyl-s-triazines. One such hydroxyphenyl-s-triazine is bemotrizinol. In still further embodiments the UV absorber can be 2-(4,6-Diphenyl-1,3,5-triazin-2-yl)-5-[(hexyl)oxy]-pheno1,4-[[4,6-bis[[4-(2-ethylhexoxy-xomethyl)phenyl]amino]-1,3,5-triazin-2-yl]amino]benzoicacid2-ethylhexyl ester (ethylhexyl triazone), 2-(2-Hydroxy-4-methoxyphenyl)-4,6-diphenyl-1,3,5-triazine,2-(4,6-Bis-(2,4-dimethylphenyl)-1,3,5-triazin-2-yl)-5-(octyloxy)-phenol, 2-[4-[2-hydroxy-3-tridecyl oxypropyl]oxy]-2-hydroxyphenyl]-4,6-bis(2,4-dimethylphenyl)-1,3,5-triazine and 2-[4-[2-hydroxy-3-dodecyl oxypropyl]oxy]-2-hydroxyphenyl]-4,6-bis(2,4-dimethylphenyl)-1,3,5-triazine (Tinuvin® 400), 2-[2-Hydroxy-4-[3-(2-ethylhexyl-1-oxy)-2-hydroxypropyloxy]phenyl]-4,6-bis(2,4-dimethylphenyl)-1,3,5-triazine (Tinuving405),642,6-bis(2,4-dimethylphenyl)-1H-1,3,5-triazin-4-ylidene]-3-(6-methylheptoxy)cyclohexa-2,4-dien-I-one,2,4-Bis(2-hydroxy-4-butyloxyphenyl)-6-(2,4-bis-butyloxyphenyl-1,3,5-triazine (Tinuvin® 460), Isooctyl 2-[4-[4,6-bis[(1,1′-biphenyl)-4-yl]-1,3,5-triazin-2-yl]-3-hydroxyphenoxy]propanoate (Tinuvin® 479), 2-(2′-hydroxy-5-methylphenyl)-5-benzotriazole, 2-(2H-benzotriazol-2-yl)-4-(1,1,3,3-tetramethylbutyl)phenol, 2-(2′-Hydroxy-3′-t-butyl-5′-methylphenyl)-5-chlorobenzotriazole,2-(2-hydroxy-3,5-di(1,1-dimethyl-benzyl)-2-benzotriazole, a-[3-[3-(2H-Benzotriazol-2-yl)-5-(1,1-dimethylethyl)-4-hydroxyphenyl]-1-oxopropyl]-m-hydroxypoly(oxo-1,2-ethanediyl),a-[3-[3-(2H-Benzotriazol-2-yl)-5-(1,1-dimethylethyl)-4-hydroxyphenyl]-1-oxopropyl]-m-[3-[3-(2H-benzotriazol-2-yl)-5-(1,1-dimethylethyl)-4-hydroxyphenyl]-1-oxopropoxy]poly(oxy-1,2-ethanediyl),2-(2H-Benzotriazol-2-yl)-4,6-bis(1-methyl-1-phenylethyl)phenol (Tinuvin® 900), 2-(2H-Benzotriazol-2-yl)-6-(1-methyl-1-phenylethyl)-4-(1,1,3,3-tetramethylbutyl)phenol (Tinuvin® 928), and combinations thereof.

A photo-stabilizer can be added to the ultraviolet light-absorbing particle according to the second aspect to inhibit photodegradation of the UV-absorber, thereby increasing the service life of the UV absorber. One such photo-stabilizer can be a hindered amine. Useful hindered amines include 2,2,6,6-tetramethylpiperidine, an alkylated or hydroxylamine analog of 2,2,6,6-tetramethylpiperidine, or a polymer containing any of these functional groups.

In advantageous embodiments of the second aspect the ultraviolet light-absorbing particle is suitable for injection into the dermal layer of the skin. The particle can be in the form of (A) Polymer particles, (B) Molecular aggregates, (C) Inorganic nano- or microparticles, (D) Surface-coated nano- or microparticles, (E) Core-shell nano- or microparticles, or (F) Mesoporous nano- or microparticles.

In further advantageous embodiments of the second aspect the ultraviolet light-absorbing particle is provided in combination with a tattooable biosensor that is sensitive to radiation, ion concentrations, pH, or glucose levels, or other measurable analyte or biomolecule as will be apparent to one of skill in the art.

In certain embodiments according to the second aspect the ultraviolet light-absorbing particle is poly(methyl methacrylate) (PMMA), polylactic acid (PLA), poly(lactic-co-glycolic acid) (PLGA), poly(dimethylsiloxane) (PDMS), polyethylene glycol (PEG), Melamine-formaldehyde, Methacrylamide chitosan.

Commercially-available UV absorbers suitable for application in ultraviolet light-absorbing particles include hydroxybenzophenone, hydroxyphenyl-s-triazine, and 2-(2-hydroxyphenyl)benzotriazole, oxalanilide, Aminobenzoic acid, Avobenzone, Cinoxate, Dioxybenzone, Homosalate, Meradimate, Octocrylene, Octinoxate, Octisalate, Oxybenzone, Padimate 0, Ensulizole, Sulisobenzone, Titanium dioxide, Trolamine salicylate, and Zinc oxide and derivatives and/or combinations thereof.

The ultraviolet light-absorbing particle according to the second aspect can include an antioxidant. Examples of useful antioxidants include polyphenols, vitamins, carotenoids, hindered phenols, phosphites, melanin or combinations thereof. With regard to polyphenols, the polyphenol can be a flavonoid, hydroxycinnamic and hydroxybenzoic acids, tannin, cucurmin, gingerol, and combinations thereof. Examples of useful vitamins include vitamins A, C, E, or combinations thereof. Examples of useful carotenoids include beta-carotene, lycopene, or combinations thereof.

In an advantageous embodiment the ultraviolet light-absorbing particle according to the second aspect can be suspended in a biocompatible solvent such as water, alcohols (e.g., ethanol, isopropanol, glycerol, oligo- and polyethylene glycols), oils (e.g., vegetable oils/triglycerides, geraniol, squalene, etc.), or combinations thereof. Examples of suitable biocompatible solvent include water, ethanol, isopropanol, glycerol, oligo- and polyethylene glycols, vegetable oils/triglycerides, geraniol, squalene, and combinations thereof.

The ultraviolet light-absorbing particle according to the second aspect can include an additive such as (i) antiseptics (e.g. alcohols) to prevent bacterial contamination, (ii) biocompatible surfactants (e.g., polysorbates) to stabilize the dispersions and adjust surface tension, (iii) thickening agents (e.g. xanthan gum, polyacrylates, polyglycols) to increase viscosity and reduce pigment sedimentation rates (iv) thixotropic agents (e.g. silica) to promote shear thinning (v) preservatives/binding agents (e.g. polyethers, polyvinylpyrrolidinone) to help prevent the inks from drying and to help them bind to needles, (vi) astringents to minimize bleeding in the skin upon implantation, (vii) anesthetics to minimize pain during ink implantation, and combinations thereof. The antiseptic can be an alcohol such as ethanol, isopropanol, glycerol, and poly(ethylene glycol). Examples of useful biocompatible surfactants include polysorbate, TWEEN-20, TWEEN-80 and poly(vinyl alcohol). Examples of useful thickening agents include is xanthan gum, polyacrylates (e.g. poly(acrylic acid) and co-polymers of poly(acrylic acid) and other acrylates including methyl acrylate, methyl methacrylate, ethyl acrylate, propyl acrylate, butyl acrylate, etc.), polyglycols (e.g. poly(ethylene glycol) and poly(propylene glycol)) or combinations thereof.

The ultraviolet light-absorbing particle according to the second aspect can include TWEEN-80 surfactant at ratio of <1.0% (v/v) to stabilize the suspension, and polyethylene glycol (molecular weight 1000) or glycerol added at a ratio of 10%-30%, whereby the polyethylene glycol or glycerol can act as an antiseptic agent, thickener, or binder.

In an advantageous embodiment, the ultraviolet light-absorbing particle according to the second aspect is in the microparticle to nano-particle size range.

In a third aspect the present invention provides a formulation of transparent or nearly transparent nanoparticles and/or microparticles (the nanoparticles or microparticles can be highly absorptive in the UVA and UVB range) in combination with a biocompatible solvent suitable for injection into the dermal or intradermal layer of the skin. The formulation according to the third aspect can include an ink or pigment suitable for dermal implantation.

In a fourth aspect the present invention provides a method of implanting an ultraviolet light-absorbing particle into the skin of a subject. The method can include the steps of (1) providing a composition comprising any one of the particles or formulations according to the first four aspects; (2) contacting the skin with a microneedle having the provided composition; and (3) penetrating the contacted skin with the microneedle. In an advantageous embodiment the microneedle is a dissolving microneedle. The dissolving microneedle can include a suitable carrier such as polyvinylpyrrolidinone or polyvinyl alcohol and their liquid pre-polymers, or aqueous solutions of carboxymethyl cellulose, trehalose, maltodextrin, galactose, glucose, and silk.

In a fifth aspect the present invention provides a second method of implanting an ultraviolet light-absorbing particle into the skin of a subject. The method can include the steps of (1) providing a composition comprising any one of the particles or formulations according to the first four aspects; (2) contacting the skin with a needle-free tattoo machines configured to deliver the provided composition in combination with a tattoo ink; and (3) penetrating the contacted skin with the composition in combination with tattoo ink droplets at sufficiently high velocity to penetrate into the dermis. A sufficiently high velocity can be a velocity that exceeds 40 m/s.

In a sixth aspect the present invention provides a third method of implanting an ultraviolet light-absorbing particle into the skin of a subject. The method can include the steps of (1) providing a composition comprising any one of the particles or formulations according to the first four aspects; (2) contacting the skin with an (electric) tattoo or permanent makeup machine (rotary or coil) configured to deliver the provided composition in combination with a tattoo ink; and (3) penetrating the contacted skin with the composition in combination with tattoo ink droplets under conditions sufficient to penetrate into the dermis.

In an advantageous embodiment, the ultraviolet light-absorbing particle according to any one of the aforementioned aspects will include a tattooable UV sensor. Examples of such tattooable UV sensors are disclosed in Butterfield, J. L., Keyser, S. P., Dikshit, K. V., Kwon, H., Koster, M. I., & Bruns, C. J. (2020). Solar Freckles: Long-Term Photochromic Tattoos for Intradermal Ultraviolet Radiometry. ACS Nano, 14(10), 13619-13628.

In a seventh aspect the present invention provides a kit for embedding the biocompatible UV-absorbing nanoparticles or microparticles, such as a particle disclosed in the aforementioned aspects, in the skin of a subject. This kit may contain the biocompatible UV-absorbing nanoparticles or microparticles in one or more vials, syringes, blister packs, or other suitable containers. The nanoparticles or microparticles may be suspended in a biocompatible solvent. The suspended particles may be provided at a concentration suitable for delivery to the skin or a subject or the suspended particles may be supplied in a concentrated form, along with instructions for mixing the particles with a suitable diluent. Additionally, the particles may be provided in a dried form (e.g. desiccated) along with a biocompatible diluent and instructions for the suspension of the particles. Embodiments of the kit may further include one or more needles to facilitate delivery of the biocompatible UV-absorbing nanoparticles or microparticles. In certain embodiments the needle is a microneedle. The microneedle can be a dissolving microneedle. The dissolving microneedle can include a suitable carrier such as polyvinyl pyrrolidinone or polyvinyl alcohol and their liquid pre-polymers, or aqueous solutions of carboxymethyl cellulose, trehalose, maltodextrin, galactose, glucose, and silk diluent, along with instructions for use of the microneedle.

As previously discussed, the kit may contain the biocompatible UV-absorbing nanoparticles or microparticles in one or more vials or other suitable containers. The kit may further include an ink or pigment suitable for dermal implantation in the same container or in a separate container. When supplied separately, the kit may include instructions for mixing the particles with the ink or pigment. The kit may include a plurality of inks or pigments to enable a user to tailor the deliverable to the skin tone or desired tattoo of the subject receiving the dermal implantation.

The kit may further include a tattooable biosensor that is sensitive to radiation, ion concentrations, pH, or glucose levels in the same container or in a separate container, along with instructions for the dermal implantation of the biosensor in combination with the biocompatible UV-absorbing nanoparticles or microparticles, and their admixture when supplied separately within the kit or kits.

In further embodiments, the biocompatible UV-absorbing nanoparticles or microparticles may be supplied in a vial or cartridge suitable for loading and subsequent delivery in a needle-free injection system.

### BRIEF DESCRIPTION OF THE DRAWINGS

For a fuller understanding of the invention, reference should be made to the following detailed description, taken in connection with the accompanying drawings, in which:

FIG. 1 is an illustration providing a graphical representation of different ultraviolet-absorptive microparticle formulations. (A) Polymer particles, (B) Molecular aggregates, (C) Inorganic nano- or microparticles, (D) Surface-coated nano- or microparticles, (E) Core-shell nano- or microparticles, (F) Mesoporous nano- or microparticles.

FIG. 2 is a set of graphs (two graphs in (A) and one in (C)) and an image (B) showing characterization data of ultraviolet-absorptive nanoparticles. (A) Size distribution data for PMMA nanoparticles prepared according to the example procedure, as well as graphitic carbon nitride nanoparticles (g-C3N4) prepared by heating melamine in a furnace at 450° C. (B) SEM micrograph of PMMA nanoparticles prepared according to the example procedure. (C) Normalized UV-Vis absorption spectrum of a dilute suspension of ultraviolet-absorptive nanoparticles made of graphitic carbon nitride.

FIG. 3 is a pair of images (labeled A and B) showing ultraviolet-absorptive nanoparticle tattoo inks. (A) Photograph of a vial of ultraviolet-absorptive microparticle tattoo ink (Formulation A), which appears cloudy white due to scattering. (B) A UV photograph (wavelength sensitivity 360-380 nm) of the same tattoo ink shows that it is “black”, or highly absorptive, in the UVA range.

FIG. 4 is a set of images comparing visible (top) and UV (bottom) photographs of an ex vivo porcine skin sample tattooed with carbon black tattoo ink, PDMS nanoparticle tattoo ink, and an ultraviolet-absorptive nanoparticle tattoo ink based on the bemotrizinol-doped PMMA nanoparticles described in the example procedure. While the tattoo is minimally visible to the naked eye, like PDMS, it is dark in the UVA range due, like carbon black, to UV absorption by the implanted ultraviolet-absorptive nanoparticles.

### DETAILED DESCRIPTION OF THE PREFERRED EMBODIMENT

Tattoos are formed using intradermal nanoparticles (typically 20-900 nm in diameter) in the form of color additives, most often borrowed from the pigment manufacturing industry. [Baumler, W., et al., Lasers Surg. Med 2000, 26, 13-21; Hogsberg, T., et al., Br. J Dermatol. 2011, 165, 1210-1218; Rubio, L., et al., Anal. Chim. Acta 2019, 1079, 59-72; Hansen, P., et al., Danish Environmental Protection Agency. 2006.] Tattoo pigments are typically inserted in the dermis by repeatedly puncturing the skin with a needle or array of needles carrying a tattoo ink comprising a dispersion of these pigments, although alternative needle-free injection strategies are taught in U.S. Patent 2012/0179134 A1 and in further development. [Oyarte Galvez, L. et al., High speed imaging of solid needle and liquid micro-jet injections. J Appl. Phys. 2019, 125, 144504-13; Cu, K.; Bansal, R.; Mitragotri, S.; Rivas, D. F. Delivery Strategies for Skin: Comparison of Nanoliter Jets, Needles and Topical Solutions. Ann. Biomed Eng. 2019, 2028-2039.] Without intervention, tattoos leave permanent markings on the skin because the pigments undergo repeated cycles of capture and release by dermal melanophages with minimal migration in the dermis. [Baranska, A et al., Unveiling Skin Macrophage Dynamics Explains Both Tattoo Persistence and Strenuous Removal. J Exp. Med 2018, 215, 1115-1133.] Tattoo fading is caused by clearance of the pigments via drainage into the lymph nodes by these immune cells and this process may be accelerated by pigment photodegradation associated with laser tattoo removal treatment as well as UV exposure in sunlight. [Engel, E. et al., JDDG 2007, 5, 583-589; Engel, E. et al., Exp. Dermatol. 2009, 19, 54-60; Gonzalez, C. D. et al., Photodermatology, Photoimmunology & Photomedicine 2020, 36, 73-74; Gonzalez, C. D. et al., J Clin. Aesthet. Dermatol. 2020, 13, 22-23.]

Although tattoos are most commonly used for body decoration, a limited number of biomedical applications for tattoos have been developed. Biomedical tattoos have been utilized in pre-surgical demarcation of anatomical biopsy sites, as well as in medical aesthetics applications such as reconstructive surgery, hair loss restoration, and resistant vitiligo. [Vassileva, S. and Hristakieva, E., Medical Applications of Tattooing. Clin. Dermatol. 2007, 25, 367-374; Jalgaonkar, A et al., Preoperative biopsy tract identification using india ink skin tattoo in tumous surgery. Orthopaedic Proceedings 2012, 94-B:SUPP_XXXVII, 321; Becker, H. The Use of Intradermal Tattoo to Enhance the Final Result of Nipple-Areola Reconstruction. Plast. Reconstr. Surg. 1986, 77, 673; Rassman, W. R. et al., Scalp Micropigmentation: A Concealer for Hair and Scalp Deformities. J Clin. Aesth. Dermatol. 2015, 8, 35-42; Tanioka, M. et al., Camouflage for patients with vitiligo vulgaris improved their quality of life. J Cosmet. Dermatol. 2010, 9, 72-75.] These applications typically rely on conventional tattoo pigments to color the skin, although some pre-biopsy tattoo pigments have been engineered to exhibit fluorescence [Chuang, G. S.; Gilchrest, B. A Dermatol. Surg. 2012, 38, 479.] and programmable intradermal retention times. [Choi, J. et al., Cross-Linked Fluorescent Supramolecular Nanoparticles as Finite Tattoo Pigments with Controllable Intradermal Retention Times. ACS Nano 2017, 11, 153-162] More recently, the concept of tattooable biosensors sensitive to ion concentrations, pH, and glucose levels has been explored in ex vivo skin models, and synthetic biology-based cellular tattoos with pigmentation sensitive to hypercalcemia have been demonstrated in vivo in mice. [Vega, K. et al., Proceedings of the 2017 ACM International Symposium on Wearable Computers 2017, 138-145; Yetisen, A K. et al., Angew. Chem. Int. Ed Engl. 2019, 58, 10506-10513; Jiang, N. et al., Fluorescent Dermal Tattoo Biosensors for Electrolyte Analysis. Sens. Actuators B Chem. 2020, 320, 128378; Tastanova, A et al., Synthetic Biology-Based Cellular Biomedical Tattoo for Detection of Hypercalcemia Associated with Cancer. Sci. Transl. Med 2018, 10, eaap8562.]

The present invention provides permanent or semi-permanent UV protection in the skin. In a first aspect the technology utilizes formulations of transparent or nearly transparent nanoparticles and/or microparticles, which are highly absorptive in the UVA and UVB range (See Example 1, below). In further aspects the present invention provides inks (See Example 2, below) utilizing dispersions of these particles, such as in the first aspect, that enable implantation in the dermis. In still further aspects the present invention provides techniques for implanting the inks in the dermis, including conventional tattooing, permanent make-up, threading, and microneedle patches (See Example 3, below).

### Example 1—Materials and Methods

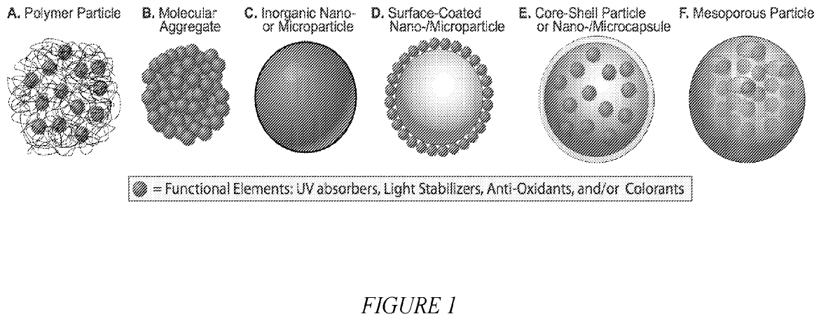

The present invention provides formulations for visibly transparent or colorless UV-absorptive particles (see e.g., FIG. 1). The mean particle diameters will advantageously fall within the range of approximately 20 nm to 10 microns in order to (i) facilitate implantation in the dermis by tattooing or other means and (ii) to remain located semi-permanently or permanently in the dermis. As particle size becomes less than the lower limit of this size range (i.e., lower than about 20 nm) the particles are more easily cleared by the immune system. On the other hand, larger particles (e.g., in excess of about 10 microns) may lead to excessive granuloma or keloid reactions. The particles can contain “functional elements”, depicted as darker spheres in FIG. 1. These functional elements can comprise, minimally, a UV absorber. By “UV absorber” it is meant as any compound that meets the following two criteria: (i) the compound absorbs a substantial amount of light in the ultraviolet wavelength range of 280-400 nm, and (ii) the compound absorbs a proportionally minimal (e.g. s10% of the absorbance in the UV range) amount of light in the visible wavelength range of approximately 400-800 nm. Light absorbance can be measured with a spectrophotometer. “Substantial” absorbance can be an absorptivity coefficient greater than 1 L/(g·cm) at a specified wavelength.

In addition to the UV absorber, the formulations may also include any combination of the following functional elements:

UV Absorbers. Additional UV absorbers may be included to tune the spectral distribution of the particles in the UV range or improve the photostability of the formulation. So, by way of nonlimiting example, tuning the spectra distribution of a given formulation can be achieved by changing the shape and intensity of the UV absorption profile over the wavelength range of 280-400 nm.

Various classes of UV absorbers are possible and appropriate for inclusion in the UV-absorptive particles. Organic UV absorbers can include FDA-approved over-the-counter sunscreen drugs, [see e.g., U.S. Food and Drug Administration. Sunscreen Drug Products for Over-the-Counter Human Use: Proposed Rule. Federal Register 2019, 84, 6204-6275] industrial additives for coatings, such as benzophenones, benzotriazoles, and phenyltriazines [as taught in U.S. Patent No. US 2006/0153783], [see e.g., Keck, J. et al., J Phys. Chem. 1996, 100, 14468-14475; Schaller, C. et al., J Coat. Technol. Res. 2007, 5, 25-31.] or polymers incorporating these moieties within their repeating units. [Huang, Z. et al., Sci. Reports 2016, 6:25508.] Inorganic/mineral UV absorbers include TiO2, [Allen, N. S. et al., Polym. Degrad Stabil. 2002, 78, 467-478.] ZnO, [Becheri, A et al., J Nanopart. Res. 2007, 10, 679-689.] doped SiO2, [He, Q. et al., J Phys. Chem. Solids 2004, 65, 395-402] CeO2, [Goubin, F. et al., Chem. Mater. 2004, 16, 662-669.] etc., which may be either crystalline, polycrystalline, or amorphous. UV absorbers can also include organic/inorganic combinations, [Mahltig, B. et al., Thin Solid Films 2005, 485, 108-114] including layered double hydroxides. [Feng, Y. et al., Polym. Degrad Stabil. 2006, 91, 789-794; Li, D. et al., J Solid State Chem. 2006, 179, 3114-3120; Cao, T. et al., RSC Advances 2013, 3, 6282-6285.]

Photo-stabilizers. In the case of small-molecule and polymer organic UV absorbers, it is often beneficial to mix them with photo-stabilizers that can inhibit photodegradation thereby increasing the service life of the UV absorber and the other materials in the particles. [Muasher, M.; Sain, M. The efficacy of photostabilizers on the color change of wood filled plastic composites. Polym. Degrad Stabil. 2006, 91, 1156-1165; Andrady, A L. et al., Effects of increased solar ultraviolet radiation on materials. J Photochem. Photobiol. B 1998, 46, 96-103.] Hindered amines, particularly those derived from 2,2,6,6-tetramethylpiperidine and its alkylated or hydroxylamine analogs, are an advantageous class of photostabilizer. These photostabilizers scavenge undesired radicals generated in organic materials under UVA and UVB irradiation and are subsequently regenerated (the Denisov cycle [Hodgson, J. L.; Coote, M. L. Clarifying the mechanism of the Denisov cycle: How do hindered amine light stabilizers protect polymer coatings from photo-oxidative degradation? Macromolecules 2010, 43, 4573-4583]), imparting them with long-lasting light stabilizing functionality. [Klemchuk, P. P.; Gande, M. E. Stabilization mechanisms of hindered amines. Polym. Degrad Stabil. 1988, 22, 241-274.]

Anti-Oxidants. Anti-oxidants, such as hindered phenols [see e.g., Klemchuk, P. P.; Horng, P. L. Transformation products of hindered phenolic antioxidants and colour development in polyolefins. Polym. Degrad Stabil. 1991, 34, 333-346] or phosphites [see e.g., Tochacek, J.; Sedlaf, J. Polym. Degrad Stabil. 1993, 41, 177-184; Habicher, W. D., et al., Macromol. Symp. 1997, 115, 93-125.], may also be added as functional elements. These functional elements provide a synergistic stabilization effect in many polymer materials by sacrificially preventing unwanted oxidative reactions from occurring in the polymer (i.e., they deactivate alkyl peroxyls and hydroperoxides). [Pospisil, J. Chemical and photochemical behaviour of phenolic antioxidants in polymer stabilization: A state of the art report, part 11. Polym. Degrad Stabil. 1993, 39, 103-115; Pospisil, J.; Nespurek, S. Photostabilization of coatings. Mechanisms and performance. Frog. Polym. Sci. 2000, 25, 1261-1335.] A number of suitable anti-oxidants may also be derived from natural sources, including polyphenols (e.g., flavonoids, hydroxycinnamic and hydroxybenzoic acids, tannin, cucurmin, gingerol), vitamins (e.g., vitamins A, C, E), and carotenoids (e.g. beta-carotene, lycopene). [Dintcheva, N. T.; D′ Anna, F. Anti-/pro-oxidant behavior of naturally occurring molecules in polymers and biopolymers: a brief review. ACS Sustainable Chem. Eng. 2019, 7, 12656-12670.] Although many of these naturally occurring compounds are not transparent in the visible region, they may be appropriate for use in small quantities such that their coloration of the particles is minimal or skin-toned. As natural melanin also exhibits anti-oxidative and anti-inflammatory effects, [ElObeid, A S. et al., Pharmacological Properties of Melanin and its Function in Health. Basic Clin. Pharmacol. Toxicol. 2017, 120, 515-522.] these elements may imbue the particles with additional health benefits akin to natural melanin.

Colorants. Since scattering may cause colorless nanoparticles or microparticles to appear white, the formulations may be mixed with colorants in the form of dyes or pigments in order to match the color of the formulation to the skin tone of the subject/patient. Biocompatible dyes or pigments may be mixed with the polymer carrier, UV absorber, and any other ingredients during the synthesis of the nano- or microparticles to render their appearance as a skin-toned color. An exemplary biocompatible pigment is melanin.

Preferably, the particles will exhibit little to no toxicity, immunogenicity, or teratogenicity. Particles will also exhibit high chemical, physical, and photo stability in aqueous media in the temperature range of 20-40° C., which is representative of intradermal conditions. Particles exhibiting these characteristics should maintain their long-term function and biocompatibility in the skin. The functional elements can also be insoluble, (or rendered insoluble by chemical or encapsulation strategies, vide infra) in aqueous media to prevent them from partitioning into the interstitial fluid. It is preferred to minimize the scattering, reflectance, and refraction of the particles in addition to their visible absorption, to minimize their visibility in skin. As scattering is highest at particle diameters near 100-200 nm,[Dawson, P. L.; Acton, J. C. Impact of proteins on food color. Proteins in Food Processing, Second Ed. 2018, Elsevier Ltd. pp. 599-638.] some preferred particle sizes are on the size scale of visible light or higher (400 nm and above). To minimize excessive reflection and refraction, which would cause the particles to appear white (Mie scattering), the refractive indices of the particles in the visible range can closely match that of the dermis (1.36-1.41[Ding, H.; et al. Refractive indices of human skin tissues at eight wavelengths and estimated dispersion relations between 300 and 1600 nm. Physics in Medicine and Biology 2006, 51, 1479-1489]). Particle formulations with excessive scattering may be made “invisible” in the skin by matching their color to the skin tone of the user with dye or pigment additives.

Formulation A Polymer Particle. The functional elements may be integrated within polymer or co-polymer particles of appropriate size (−20-10,000 nm) by a number of strategies, which may be broadly classified into dispersion approaches and polymerization approaches. [Rao, J. P.; Geckeler, K. E. Polymer nanoparticles: Preparation techniques and size-control parameters. Frog. Polym. Sci. 2011, 36, 887-913.] Dispersion approaches involve converting pre-formed polymers into nano- or microparticles from a homogenous solution by solvent evaporation in a spray or emulsion, or by precipitation with solvent exchange, salt, dialysis, or supercritical fluids. Dissolving the functional elements in the polymer phase during these processes will incorporate them (non-covalently) into the polymer matrix of the resulting nano- or micro-particles. Polymerization approaches to polymer particle synthesis typically rely on emulsions, in which nano- or micro-droplets of pre-polymer resins (monomers), typically dispersed in aqueous solutions, are directly polymerized into particles upon initiation of the polymerization. In this case, the functional elements may be dissolved into the monomer phase of the emulsion to incorporate them into the polymer matrix upon polymerization. A polymerization approach that can be applied to UV absorptive nanoparticles for aqueous dispersions is taught in Japan patent JP 6129146. In both dispersion and polymerization approaches, the functional elements may also be incorporated directly into the main chain, side chain, or cross-links of the polymer structure by including them as monomers during polymer synthesis. In most cases, the functional elements could be modified with reactive functional groups in order to be covalently bound to the polymer or co-polymer. For example, functionalizing a benzophenone, benzotriazole, or phenyltriazine-based UV absorber with one or more acrylic or vinyl functional groups would enable its polymerization or co-polymerization by catalysis or radical polymerization. Alternatively, the functional elements may be coupled to a pre-synthesized polymer [Huang, Z. et al., Sci. Reports 2016, 6:25508]. These covalent-attachment methods of incorporating functional elements are more expensive than the admixture approaches, but they lower the risk of any functional elements leaching out of the particles.

Advantageous polymer matrices in this formulation include poly(dimethylsiloxane) (PDMS) and other silicone rubbers, or poly(methyl methacrylate) (PMMA) and other methacrylate compounds (e.g., poly(methyl methacrylate, poly(isopropyl methacrylate), poly(isobutyl methacrylate)). These polymer matrices are particularly appropriate for application as a UV-adsorptive particle because (i) their biocompatibility is well-established, (ii) their refractive indices of less than 1.5 is close to that of the dermis, (iii) they exhibit high long-term stability and (iv) they are relatively convenient and inexpensive to produce. [Rahimi, A; Mashak, A Review on rubbers in medicine: natural, silicone and polyurethane rubbers. Plastics, Rubber and Composites 2013, 42, 223-230; Frazer, R. Q. et al., PMMA: An Essential Material in Medicine and Dentistry. Journal of Long-Term Effects of Medical Implants 2005, 15, 629-639.] Formulation B. Molecular Aggregate. Small-molecule or oligomer functional elements that form solids at biological temperatures may be employed directly as aggregated particles when they are sufficiently insoluble in aqueous media and of sufficient size for dermal implantation. The processes of rendering poorly water-soluble compounds into small particulates are known as nanosizing [Kesisoglou, F. et al., Nanosizing-Oral formulation development and biopharmaceutical evaluation. Adv. Drug Deliv. Rev. 2007, 59, 631-644] or micronizing. [Rasenack, N. and Muller, B. W. Micron-Size Drug Particles: Common and Novel Micronization Techniques. Pharm. Dev. Technol. 2004, 9, 1-13] Molecular aggregates can be prepared as nano- or microparticles by (i) precipitation from a solvent into a non-solvent (ideally water),[Rabinow, B. E. Nanosuspensions in drug delivery. Nat. Rev. Drug Discov. 2004, 3, 785-796] (ii) spray-drying processes, [Vehring, R. Pharmaceutical Particle Engineering via Spray Drying. Pharm. Res. 2007, 25, 999-1022] (iii) supercritical fluid techniques, [Martin, A and Cocero, M. J. Micronization processes with supercritical fluids: Fundamentals and mechanisms. Adv. Drug Deliv. Rev. 2008, 60, 339-350] or (iv) milling. [Merisko-Liversidge, E. et al., Nanosizing: a formulation approach for poorly-water-soluble compounds. Eur. J Pharm. Sci. 2003, 18, 113-120.] These methods can be used to generate nano- or microparticulate UV absorbers or mixtures of functional elements comprising organic molecules. An exemplary material of this form is micronized hydroxyphenyl-s-triazine. Another exemplary material is graphitic carbon nitride.

Formulation C. Inorganic Particles. A variety of semiconducting metal oxides may be employed as UV absorbers. [Fajzulin, I. et al., Nanoparticulate inorganic UV absorbers: a review. J Coat. Technol. Res. 2015, 12, 617-632] In contrast with organic materials, these materials do not photodegrade. TiO₂ and ZnO are among the most common UV absorbers in over-the-counter sunscreens, and they are also commonly employed as color additives in tattoo inks for their whitening effect. The UV absorptivity of these materials increases with decreasing particle size, and dominates over scattering below diameters of 50 nm. [Egerton, T. A and Tooley, I. R. UV absorption and scattering properties of inorganic-based sunscreens. Int. J Cosmet. Sci. 2011, 34, 117-122] Therefore, these materials may be employed as intradermal UV absorbers at small (<50 nm) particle sizes. However, their highly scattering and reflective properties in the visible wavelengths [Cole, C. et al., Metal oxide sunscreens protect skin by absorption, not by reflection or scattering. Photoderm. Photoimmunol. Photomed 2015, 32, 5-10.]—due to their high refractive indices (2.6 for TiO₂ and 1.9 for ZnO)—can lead to skin whitening. This issue may be addressed by employing additional tattoo pigment color additives to match the skin tone when using inorganic particles as UV absorbers. However, TiO2 and ZnO also exhibit photocatalytic activity, [Egambaram, 0. P.; Kesavan Pillai, S.; Ray, S. S. Materials Science Challenges in Skin UV Protection: A Review. Photochem. Photobiol. 2020, 36, 1345-1264] generating damaging reactive oxygen species. While this gives them with a bacteriocidal effect on the skin's surface, it may lead to tissue and DNA damage intradermally. Alternative inorganic UV absorbers may also be employed (e.g., CeO₂, Fe₂O₃), they likely face the similar issues, since UV absorptivity arises from the semiconductor bandgap, yet photocatalysis occurs when semiconductors absorb energy greater than their bandgap. If inorganic particles are to be used as the UV absorbers, they can be employed with surface coatings, such as those described as in Formulations D and E, below, to prevent photocatalysis.

Formulation D. Surface-Coated Particle. A monolayer or multilayer of UV absorbers and other functional elements can be adsorbed to the surface of a nano- or microparticle by chemical or physical means. Covalent attachment of the functional elements to the particle affixes the UV absorber to the particle surface. For example, a surface-coated particle can employ silica particles as the substrate. Silica is an appropriate material because (i) it is already employed as a thixotropic agent in tattoo inks [Piccinini, P. et al., Safety of tattoos and permanent make-up: Final report. European Commission Joint Research Centre Science for Policy Report 2016, 1-118] and it can be biocompatible, (ii) it is readily functionalized by silanization [Voort, Der, P. V.; Vansant, E. F. Silylation of the Silica Surface A Review. J Liq. Chromatogr. R. T 2006, 19, 2723-2752.] with a wide variety of alkoxysilanes and halosilanes. The functional elements would need to be modified to display these silane functional groups for covalent attachment to SiO₂. Polymer particles may also be formulated for surface modification, provided they display reactive functional groups that can be coupled to the functional elements. However, due to the low mass and volume ratio of functional elements in this formulation, it is expected to be less effective than Formulations E and F, presented below.

Formulation E. Core-shell Particle. Core-shell particles include formulations of core fluid/polymer shell, core fluid/inorganic shell, core polymer or gel/polymer shell, and core polymer or gel/inorganic shell. A convenient inorganic shell in this formulation is silica because it renders inorganic particles more biocompatible. [Gerion, D. et al., Synthesis and properties of biocompatible water-soluble silica-coated CdSe/ZnS semiconductor quantum dots. J Phys. Chem. B 2001, 105, 8861-8871.] The core or shell polymers may constitute the same polymers as discussed in Formulation A, with PDMS and PMMA being preferred for their transparency and biocompatibility. Core-shell particles are also known as nanocapsules or microcapsules, especially when they contain fluid cores, and they may be produced by a variety of emulsion-polymerization techniques, [Jamekhorshid, A. et al., A review of microencapsulation methods of phase change materials (PCMs) as a thermal energy storage (TES) medium. Renew. Sust. Energy Rev. 2014, 31, 531-542] as well as by microfluidic reactor approaches [Wang, J.-T. et al., Fabrication of Advanced Particles and Particle-Based Materials Assisted by Droplet-Based Microfluidics. Small 2011, 7, 1728-1754.] and spray drying. [Gharsallaoui, A. et al., Applications of spray-drying in microencapsulation of food ingredients: An overview. Food Research International 2007, 40, 1107-1121.]

Formulation F. Mesoporous Silica Nanoparticles. Mesoporous silica nanoparticles (MSNPs) are highly developed as nanocarriers for drug delivery applications. [Slowing, I. I. et al., Mesoporous silica nanoparticles as controlled release drug delivery and gene transfection carriers. Adv. Drug Deliv. Rev. 2008, 60, 1278-1288.] Their widespread use and biocompatibility in many settings make them, likewise, attractive carriers for UV absorbers and other functional elements of absorptive microparticles. [Asefa, T.; Tao, Z. Biocompatibility of mesoporous silica nanoparticles. Chem. Res. Toxicol. 2012, 25, 2265-2284; Tam, D. et al., Mesoporous silica nanoparticle nanocarriers: biofunctionality and biocompatibility. Acc. Chem. Res. 2013, 46, 792-801] However, in contrast with drug delivery, where the contents of the particle are meant to be released, the functional elements must be permanently contained in the case of ultraviolet-absorptive microparticles. Therefore, an advantageous method is to covalently attach the functional elements to the SiO2 surface using alkoxysilanes and halosilanes. [Voort, Der, P. V. et al., J Liq. Chromatogr. R. T 2006, 19, 2723-2752] However, it is also possible to contain the functional elements within the pores as long as the pore openings at the surface are sufficiently blocked to eliminate mass transport (cargo release). An advantage of MSNPs over silica nanoparticles (Formulation D) is that their much higher surface area (which can exceed 1000 square meters per gram) allows for a higher density of functional elements to be adsorbed to the surface of each particle (ultimately leading to more potent UV-absorptive inks and tattoos).

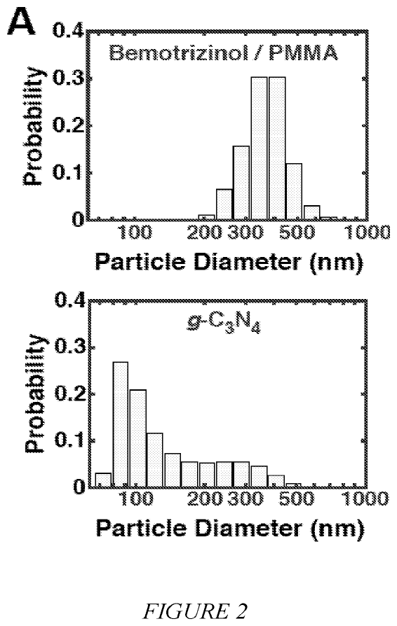

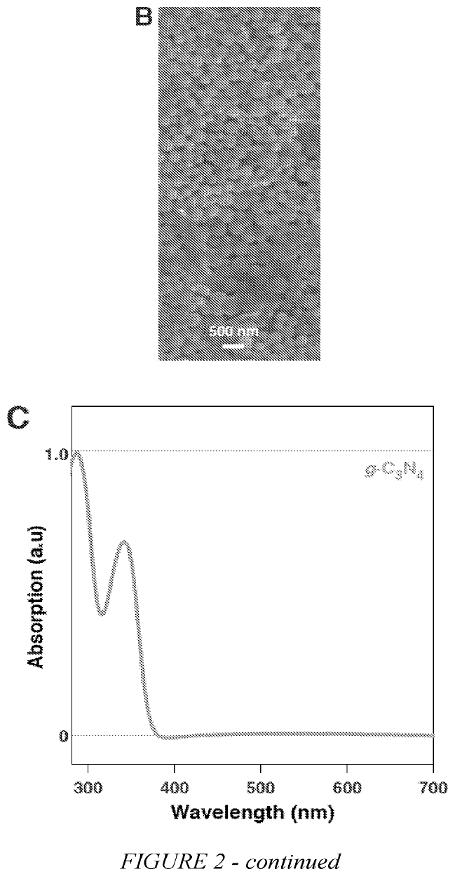

Example procedure for the preparation of nano- or microparticles: Ultraviolet-absorptive nanoparticles of Formulation A, comprising a PMMA matrix doped with bemotrizanol (marketed by BASF as Tinosorb® S) as a UV absorber. 100 mg of PMMA (35,000 Da) and 25 mg bemotrizinol were dissolved in 4 ml of dichloromethane. This organic solution was added to an aqueous solution of polyvinyl alcohol (PVA) at a concentration of 1% m/v. The mixture was shaken by hand to form an emulsion and then this emulsion was sonicated with a horn sonicator (Branson) at room temperature for 10 minutes. The emulsion was transferred to a beaker with a stir bar and stirred at −1000 rpm at room temperature to allow the organic solvent to evaporate. After 6 hours, the suspension was transferred to a centrifugation tube. The particles were rinsed over several cycles of centrifugation, decanting the supernatant, and re-filling with purified water. The size distribution (FIG. 2A) of the particles was estimated by dynamic light scattering using a Nanotrac FLEX particle size analyzer (Microtrak), particle shape was observed (FIG. 2B) by scanning electron microscopy, and their absorption data (FIG. 2C) were collected using a Cary 5000 UV-Vis-NIR spectrophotometer (Agilent).

### Example 2—Ultraviolet-Absorptive Nanoparticle or Microparticle Inks

The ultraviolet-absorptive nanoparticles such as those described in Example 1, above, may be dispersed in solvents to prepare inks. The ink formulations may be tailored for an intradermal delivery method, such as that described below, which can include a variety of tattooing/permanent makeup methods and microneedle or needle patches.

Tattoo and Permanent Make-Up Inks. In order to generate a liquid ink suitable for dermal implantation, the ultraviolet-absorptive particles are suspended in a fluid with or without additives. An exemplary fluid is water, although other biocompatible solvents such as alcohols (e.g., ethanol, isopropanol, glycerol, oligo- and polyethylene glycols) or oils (e.g., vegetable oils/triglycerides, geraniol, squalene, etc.) may also be employed. Appropriate additives for these inks include (i) antiseptics (e.g. alcohols) to prevent bacterial contamination, (ii) biocompatible surfactants (e.g., polysorbates) to stabilize the dispersions and adjust surface tension, (iii) thickening agents (e.g. xanthan gum, polyacrylates, polyglycols) to increase viscosity and reduce pigment sedimentation rates [Petersen, H.; Roth, K. To Tattoo or Not to Tattoo? Chem. Unserer Zeit 2016, 50, 44-66] (iv) thixotropic agents [Piccinini, Pet al., Safety of tattoos and permanent make-up: Final report. European Commission Joint Research Centre Science for Policy Report 2016, 1-118.] (e.g. silica) to promote shear thinning (v) preservatives/binding agents (e.g. polyethers, polyvinylpyrrolidinone, PVA) to help prevent the inks from drying and to help them bind to needles, (vi) astringents to minimize bleeding in the skin upon implantation, and/or (vii) anesthetics to minimize pain during ink implantation. The resulting inks can be sterilized with gamma radiation or by other means, such as autoclave, heat, UV radiation, X-Ray radiation, or treatment with ethylene oxide prior to packaging and storage. The ultraviolet-absorptive microparticles may be stored after synthesis as a wet or dry slurry.

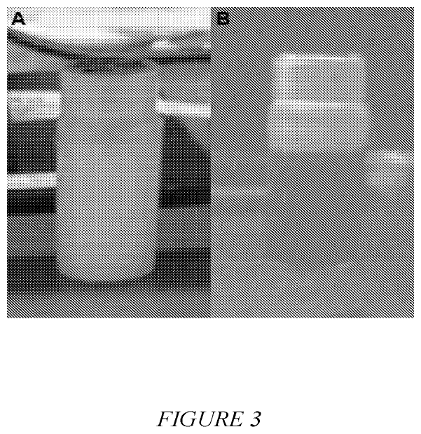

Example procedure for the preparation of ultraviolet-absorptive nanoparticle inks. A tattoo ink of an ultraviolet-absorptive microparticle of Formulation A was created by suspending the wet slurry in reverse osmosis purified water at a mass ratio of 25%. The suspension was vigorously shaken by hand in a scintillation vial for 30 seconds. The ink was characterized (FIG. 3) by photography and UV photography. The ink remained well-dispersed on the hour time-scale. Although not employed in this example, an advantageous formulation includes glycerol or poly(ethylene glycol) added at a ratio of 10%-30% as an antiseptic agent, thickener, and binder. These additives may improve the stability and transferability of the ultraviolet-absorptive nanoparticle or microparticle ink.

Microneedle Tattoo Inks. An emerging technology that should prove suitable for delivering materials, such as the ultraviolet-absorptive microparticle ink, into the dermis is the microneedle patch, a type of device with many possible configurations of micro-structured protrusions that penetrate the epidermis, which is typically targeted for transdermal drug delivery and vaccine applications. [Prausnitz, M. R. Engineering Microneedle Patches for Vaccination and Drug Delivery to Skin. Annual Rev. Chem. Biomol. Eng. 2017, 8, 177-200] U.S. Pat. No. 6,565,532 BI teaches a microneedle apparatus used for marking skin and for dispensing semi-permanent subcutaneous makeup. While these devices have not appeared on the market, it may be possible to use them for intradermal implantation of UV absorptive nanoparticles or microparticles. The ink formulations for these microneedle patches will consist of a suspension of UV absorptive nanoparticles or microparticles in a fluid containing polymer, pre-polymer, or molecular precursors to the matrix of the microneedle delivery method. For example, a formulation would employ dissolving microneedle arrays, since this formulation of microneedle patches is optimized for delivering relatively high amounts of material compared to other microneedle patch formulations. [Bediz, B. et al., Dissolvable Microneedle Arrays for Intradermal Delivery of Biologics: Fabrication and Application. Pharm. Res. 2013, 31, 117-135] The carrier matrix for dissolving microneedle arrays is advantageously a non-toxic material of sufficient strength to penetrate the epidermis, but sufficiently water soluble to dissolve rapidly in the interstitial fluid of the dermis and thus release its contents. Examples of suitable carriers for microneedle invisible ultraviolet-absorptive nanoparticle or microparticle inks include polyvinylpyrrolidinone or polyvinyl alcohol and their liquid pre-polymers, or aqueous solutions of carboxymethyl cellulose, trehalose, maltodextrin, galactose, glucose, and silk, which solidify within microneedle molds upon curing or drying, respectively.

Microneedles having needle width and depth dimensions <Imm may be too small for implanting materials permanently in the dermis, since it has a mean thickness of −2 mm and can reach thicknesses up to 4 mm, [Oltulu, P. et al., Measurement of epidermis, dermis, and total skin thicknesses from six different body regions with a new ethical histometric technique. Turk. J Plast. Surg. 2018, 26, 56-61.] and tattoo machines penetrate up to 4 mm into the skin. [Petersen, H.; Roth, K. To Tattoo or Not to Tattoo? Chem. Unserer Zeit 2016, 50, 44-66.] Dissolving needles of larger dimensions (e.g., >1 mm) could be prepared by similar methods, using masters and molds with larger-scale features, and may be more suitable for use in applications as proposed in the present invention.

### Example 3—Implantation Methods for Ultraviolet-Absorptive Microparticle Tattoos

Ultraviolet-absorptive nanoparticle or microparticle “tattoos” may be implanted by a variety of methods, typically involving a needle or array of needles, dipped in invisible ultraviolet-absorptive nanoparticle or microparticle dispersions (see Example 2, above). The ink-coated needles can repeatedly puncture the skin in order to break through the epidermal barrier and deliver the ink material into the dermis. Inserting the needle or needles into the skin may be performed by hand according to a number of ancient indigenous tattooing traditions, including tapping (tatau, Polynesia), raking (tebori, Japan), threading/stitching with needle and thread (North America), and laceration followed by ink rubbing (Europe). [Krutak, L.; Deter-Wolf, A (Eds.). Ancient Ink: The Archaeology of Tattooing 2017. Seattle; London: University of Washington Press.] An advantageous method is to attach a needle array to a modern motorized tattoo or permanent make-up machine, which improves efficiency and minimizes pain compared to hand-driven methods. Needle-free tattoo machines that inject tattoo ink droplets into the skin at sufficiently high velocity to penetrate into the dermis have been described [Garitano, G.; Garitano, L. Needleless permanent makeup and tattoo device. U.S. Pat. No. 6,689,095 B1. (2004, February 10).]. To the extent compatible with standard tattoo inks, these machines may also be employed in the present application.

Alternatively, the ink may be formulated into a dissolving microneedle or needle patch in a PDMS mold as described by Bediz et al. [Bediz, B.; Korkmaz, E.; Khilwani, R.; Donahue, C.; Erdos, G.; Falo, L. D., Jr; Ozdoganlar, 0. B. Dissolvable Microneedle Arrays for Intradermal Delivery of Biologics: Fabrication and Application. Pharm. Res. 2013, 31, 117-135]. A patch can be employed that is inserted in the skin only once and held in place for sufficient time to allow the UV-absorptive particles to be released in the interstitial fluid of the dermis.

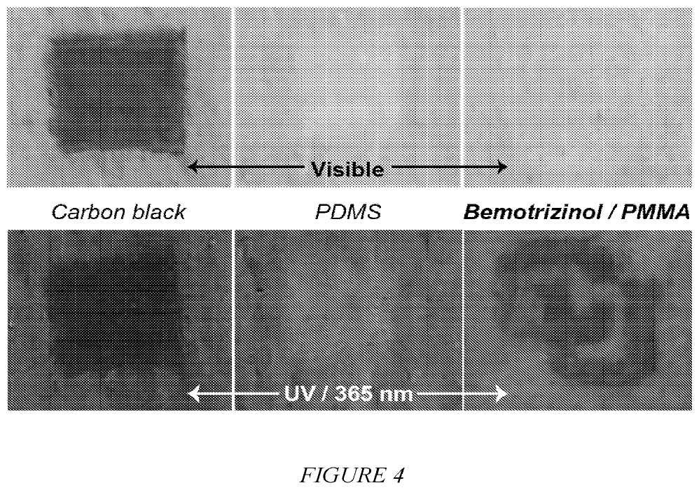

Example procedure for implantation of ultraviolet-absorptive microparticle inks. Using an ex vivo porcine skin model, an invisible ultraviolet-absorptive nanoparticle tattoo was implanted with a rotary tattoo machine (Dragonhawk) equipped with a steel 9RS tattoo needle array, dipped in an aqueous dispersion of approximately 25 wt % PMMA-based invisible ultraviolet-absorptive nanoparticles (described in the Examples 1 and 2, above) at a drive power of 7 V over an area of 1 square centimeter until a tattoo with a “hidden” UV-absorptive design of uniform appearance was obtained. The skin sample was cleaned with isopropanol before and after tattooing. Photographs of this invisible ultraviolet-absorptive particle tattoo in the visible and UVA range are shown in FIG. 4 and compared with UV-absorptive carbon black and UV-transparent PDMS nanoparticles, verifying that the tattoos based on the UV-absorptive bemotrizinol/PMMA nanoparticles are UV absorptive in the skin.

### Example 4—Applications of Ultraviolet-Absorptive Nanoparticle or Microparticle Tattoos

Uses and Benefits of the Innovation. Ultraviolet-absorptive nanoparticle or microparticle tattoos may be used to lower an individual's risk of UV-induced skin cancer, to protect against and manage symptoms of other UV-associated skin disorders and complications, to reduce skin damage and aging associated with UV exposure, to help preserve and protect pigment tattoos and tattooed skin, to modulate the sensitivity of intradermal UV radiometers and UV dosimeters, or to create invisible markings on the skin that can be detected only with a UV camera, such as described below.

Skin Cancer Risk Mitigation. By absorbing UV light that would otherwise backscatter and be absorbed by genes and tissues, ultraviolet-absorptive nanoparticle or microparticle tattoos will reduce the harmful effects of UV radiation that make UV radiation the leading risk factor for skin cancers. Black pigment tattoos exhibit a significant anti-photocarcinogenic effect in mice, likely due to a UV-absorption mechanism. [Lerche, C. M. et al., Black tattoos protect against UVR-induced skin cancer in mice. Photoderm. Photoimmunol. Photomed 2015, 31, 261-268.] The present ultraviolet-absorptive nanoparticle or microparticle tattoo technology offers a similar or superior level of protection from UV-induced skin cancer without significantly altering skin coloration.

Symptom Management in Other UV-Related Skin Complications. A number of other skin conditions and autoimmune diseases are associated with UV exposure.

Inflammation/“Sunburn”: UVB irradiation causes “sunburn” (erythema) by triggering a cascade of cytokines, vasoactive and neuroactive mediators that cooperatively produce in an inflammatory response in the skin. If the UVB dose exceeds a certain threshold, dependent on melanin density and other genetic factors, keratinocytes apoptose and die. [Clydesdale, G. J. et al., Ultraviolet light induced injury: Immunological and inflammatory effects. Immunol. Cell. Biol. 2001, 79, 547-568; Matsumura, Y.; Ananthaswamy, H. N. Toxic effects of ultraviolet radiation on the skin. Toxicol. Appl. Pharmacol. 2004, 195, 298-308.]

Photodermatoses: The most common photodermatosis is polymorphic light eruption, typically expressing as papules in UV-exposed areas. [Kang, S. et al., Fitzpatrick's Dermatology, 9e. McGraw-Hill Education, 2019.] Actinic prurigo, chronic actinic dermatitis, and acne aestivalis are less common forms of UV-induced popular or nodular eruptions. Actinic dermatitis symptoms are similar to eczema, but caused by UV exposure. Patients infected with HIV are at increased risk of experiencing these photosensitivities. Solar urticaria is a rare condition in which hives or wheals form on UV-exposed skin. Hydroa vicciniforme is another rare condition involving rashes that mature into vesicular eruptions and lead to scarring in sun-exposed skin, especially the face and hands.

Phototoxicity & Photoallergy: Acute phototoxicity occurs within hours of contact with an appropriate phototoxic agent and sufficient UV light, creating stinging or burning sensations, which may be followed by erythema and edema, and itching (pruritis), as well as vesicles or bullae in severe cases. [Kang, S. et al., Fitzpatrick's Dermatology, 9e. McGraw-Hill Education, 2019.] Pseudoporphyria occurs in severe cases and involves blisters and skin fragility. Phytophotodermatitis is also caused by UV exposure after contact with phototoxic compounds found in plants. Photoallergies may result in itchy eczema-like eruptions that are typically indistinguishable from contact dermatitis.

Favre-Racouchot syndrome: Comedones are widened openings for hair follicles and sebaceous glands filled with materials that occur in skin damaged by sunlight, especially near the eyes, in patients with this syndrome. [Paganelli, A et al., Favre-Racouchot disease: systematic review and possible therapeutic strategies. J Eur. Acad Dermatol. Venereal. 2018, 33, 32-41.]

Dermatomyositis: Women with the autoimmune disease myositis are advised to exercise extreme caution with UV exposure because it increases their probability of developing dermatomyositis, [Love, L. A; Weinberg, C. R.; McConnaughey, D. R.; Oddis, C. V.; Medsger, T. A, Jr.; Reveille, J. D.; Arnett, F. C.; Targoff, I. N.; Miller, F. W. Ultraviolet radiation intensity predicts the relative distribution of dermatomyositis and anti-Mi-2 autoantibodies in women. Arthritis Rheum.—US 2009, 60, 2499-2504.] an autoimmune disease which can lead to rashes and bumps on the face, eyelids, joints, chest, and back. Lupus erythematosus: Up to 93% of patients with the autoimmune disease lupus erythematosus experience UV photosensitivity, leading to symptoms such as erythema, inflammatory lesions, and severe skin inflammation. [Wolf, S. J. et al., Human and Murine Evidence for Mechanisms Driving Autoimmune Photosensitivity. Front. Immunol. 2018, 9, 699-12.]

The symptoms of the above conditions may be lessened, delayed, or prevented by intradermal UV absorptive particles because it reduces the effective dose of UV exposure that is experienced by the skin anatomy in sunlight.

Reducing Skin Aging: The accelerating effect of UV exposure on skin aging (including loss of tension and elasticity, and increased furrows, wrinkles, and lesions) due to photodamage and photosensitization is well-known and understood. [Rittie, L.; Fisher, G. J. UV-light-induced signal cascades and skin aging. Ageing Research Reviews 2002, 1, 705-720; Svobodova, A et al., Ultraviolet light induced alteration to the skin. Biomed Pap. Med Fae. Univ. Palacky Olomouc CzechRepub. 2006, 150, 25-38; Farage, M. A et al., Intrinsic and extrinsic factors in skin ageing: a review. Int. J Cosmet. Sci. 2008, 30, 87-95.] Ultraviolet-absorptive particles will reduce the probability of aging caused by photodamage and photosensitization events occurring within the dermis where UV light is present, and to a lesser extent in other tissue layers such as the epidermis by preventing UV light from backscattering into those regions, by absorbing and dissipating the energy of UV light with high efficiency.

Preservation of Tattoo Pigments and Tattooed Skin: UV exposure accelerates tattoo fading [Gonzalez, C. D.; Rundle, C. W.; Pona, A; Walkosz, B. J.; Dellavalle, R. P. (2020). Ultraviolet radiation may cause premature fading of colored tattoos. Photodermatology, Photoimmunology & Photomedicine, 36, 73-74]. Ultraviolet-absorptive particles as taught herein can be used as an additive in tattoo inks, or implanted on top of an existing tattoo, or applied to an area of skin before a pigment tattoo is applied. Application in such manners allows the ultraviolet-absorptive particles to act as a photo-stabilizing pigment preservative in the skin. This application of ultraviolet-absorptive particles yields colored tattoos that fade less rapidly over time.

Furthermore, tattoos occasionally lead to photodermatoses, photodermatitis, and phototoxicity. [Anderson, R. R. Shedding Some Light on Tattoos? Photochem. Photobiol. 2004, 80, 155-3; Kazandjieva, J.; Tsankov, N. Tattoos: dermatological complications. Clin. Dermatol. 2007, 25, 375-382; Khunger, N. et al,. Complications of tattoos and tattoo removal: stop and think before you ink. J Cutan. Aesthet. Surg. 2015, 8, 30-36; Vangipuram, R. and Mask-Bull, L. Histopathologic Reaction Patterns in Decorative Tattoos. J Pigment. Disord 2016, 3, 1000232; Kim, S. Y. et al., Evaluation of phototoxicity of tattoo pigments using the 3 T3 neutral red uptake phototoxicity test and a 3D human reconstructed skin model. Toxicology in Vitro 2020, 65, 104813.] Many pigments in tattoos can generate deleterious singlet oxygen when irradiated. [Regensburger, J. et al., Tattoo inks contain polycyclic aromatic hydrocarbons that additionally generate deleterious singlet oxygen. Exp. Dermatol. 2009, 19, e275-e281; H0gsberg, T. et al., Black tattoo inks induce reactive oxygen species production correlating with aggregation of pigment nanoparticles and product brand but not with the polycyclic aromatic hydrocarbon content. Exp. Dermatol. 2013, 22, 464-469.] Ultraviolet-absorptive particles as taught herein will reduce the phototoxicity in these cases by absorbing UV light that would otherwise be absorbed by the tattoo pigments and lead to these phototoxic effects.

Tuning Sensitivity of Intradermal Radiometers & Dosimeters: UV-photochromic tattoo pigments can serve as long-term intradermal UV radiometers and dosimeters. [Butterfield, J. L.; Keyser, S. P.; Dikshit, K. V.; Kwon, H.; Koster, M. I.; Bruns, C. J. Solar Freckles: Long-Term Photochromic Tattoos for Intradermal Ultraviolet Radiometry. Acs Nano 2020, 14, 13619-13628.] Admixtures of these pigments and ultraviolet-absorptive particles will reduce the effective UV irradiance reaching the photochromic pigments in a concentration-dependent manner, allowing one to fine-tune the sensitivity of these emerging intradermal sensors.

Invisible Tattoos Detectable Only by UV Camera: Since the UV-absorptive particles are not visible in the skin to the naked eye, but are visible to a UV camera, the UV-absorptive particles taught herein could be used to create hidden markings on the skin that can only detected with a UV camera. This application of the tattoo ink may be used to write hidden or encoded messages in the skin for authentication purposes, such as authentication of membership in an organization, authentication of body art, authentication of medical records, or authentication of a previous experience.

### Definitions

The term “administration” and variants thereof (e.g., “administering” a compound, “administering” a recombinant myxoma virus) in reference to a compound of the invention means introducing the compound into the system of the subject in need of treatment, such as via injection into the dermal layer of the skin of the subject. When a compound of the invention is provided in combination with one or more other active agents, “administration” and its variants are each understood to include concurrent and sequential introduction of the compound and other agents.

As used herein, the term “composition” is intended to encompass a product comprising the specified ingredients in the specified amounts, as well as any product which results, directly or indirectly, from combination of the specified ingredients in the specified amounts. A “pharmaceutically acceptable” component is one that is suitable for use with humans
and/or animals without undue adverse side effects (such as toxicity, irritation, and allergic response) commensurate with a reasonable benefit/risk ratio.

A “safe and effective amount” refers to the quantity of a component that is sufficient to yield a desired therapeutic response without undue adverse side effects (such as toxicity, irritation, or allergic response) commensurate with a reasonable benefit/risk ratio when used in the manner of this invention.

An ultraviolet light-absorbing particle is “suitable for injection into the dermal layer of the skin” when the particle is in the size range of 20 nm to 10 μm, is chemically and photochemically stable (resistant to degradation), and does not exhibit undue adverse side effects (such as toxicity, irritation, and allergic response) commensurate with a reasonable benefit/risk ratio.

As used throughout the entire application, the terms “a” and “an” are used in the sense that they mean “at least one”, “at least a first”, “one or more” or “a plurality” of the referenced components or steps, unless the context clearly dictates otherwise. For example, the term “a cell” includes a plurality of cells, including mixtures thereof.

The term “and/or” wherever used herein includes the meaning of “and”, “or” and “all or any other combination of the elements connected by said term”.

The term “about” or “approximately” as used herein means within 20%, preferably within I 0%, and more preferably within 5% of a given value or range.

As used herein, the term “comprising” is intended to mean that the products, compositions and methods include the referenced components or steps, but not excluding others. “Consisting essentially of” when used to define products, compositions and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

A “UV-absorber”, or ultraviolet light absorber, is a material that is used to dissipate ultraviolet light (i.e., electromagnetic radiation of a wavelength shorter than that of the violet end of the spectrum, having wavelengths of within the range of 4-400 nanometers, including light in the UV-A, UV-Band/or UV-C range of the spectrum) into a lower energy state.

Ultraviolet A (UVA) ultraviolet radiation with wavelengths between 320 and 400 nm, comprising over 99 percent of such radiation that reaches the surface of the earth. Ultraviolet A enhances the harmful effects of ultraviolet B radiation and is also responsible for some photosensitivity reactions; it is used therapeutically in the treatment of a variety of skin disorders.

Ultraviolet B (UVB) ultraviolet radiation with wavelengths between 290 and 320 nm, comprising less than I percent of the ultraviolet radiation that reaches the earth's surface. Ultraviolet B causes sunburn and a number of damaging photochemical changes within cells, including damage to DNA, leading to premature aging of the skin, premalignant and malignant changes, and a variety of photosensitivity reactions; it is also used therapeutically for treatment of skin disorders.

Ultraviolet C (UVC) ultraviolet radiation with wavelengths between 200 and 290 nm.

“Commercially available” means the ingredient, component or other input (e.g., UV absorber) can be purchased through a third-party supplier in an appropriate form, quality and quantity to be feasibly and economically used to fulfill an essential function (e.g., in a system employing a UV-absorber where the UV absorber to dissipates energy associated with UV light).

A “photostabilizer”, or photo-stabilizer, is a compound that helps to prevent UV absorbers or UV filters from losing their effectiveness as a result of exposure to UV radiation. Some photostabilizers help to stabilize UV absorber molecules structurally and geometrically through electrostatic and van der Waals interactions, which makes them less likely to take part in chemical reactions. Another type of photostabilizer protects a UV absorber filters by dissipating the energy from UV more rapidly, thus reducing or even eliminating the possibility of a chemical reaction. This process is called energy transfer, and it can take place when the UV absorber and photostabilizer molecules exchange electrons. In this way, the UV absorbers are freed up to do their job of protecting the skin by absorbing the harmful rays, while the photosta-bilizers do the work of disposing of the resultant energy.

Biocompatibility is a term describing the property of a material being compatible with living tissue. Biocompatible materials (e.g., biocompatible polymers, biocompatible UV absorbers, biocompatible solvents, etc.) do not produce a toxic or immunological response with living tissue or a living system, such as when exposed to the body or bodily fluids, by not being toxic, injurious, or physiologically reactive and not causing severe immunological rejection.

A biosensor is a compound or device that measures biological or chemical reactions within a biological system by generating signals responsive to the detection or the presence of an analyte or family of analytes. The generated signal is often proportional to the concentration of an analyte in the reaction.

Antioxidants are compounds or substances that inhibit or delay the oxidation of
biologically relevant molecules either by specifically quenching free radicals or by chelation of redox metals. Free radicals are produced during the biological oxidation reaction.

Kits for practicing the methods of the invention are further provided. By “kit” is intended any manufacture (e.g., a package or a container) comprising at least one reagent, e.g., a pH buffer of the invention. The kit may be promoted, distributed, or sold as a unit for performing the methods of the present invention. Additionally, the kits may contain a package insert describing the kit and methods for its use. Any or all of the kit reagents may be provided within containers that protect them from the external environment, such as in sealed containers or pouches.

The advantages set forth above, and those made apparent from the foregoing description, are efficiently attained. Since certain changes may be made in the above construction without
departing from the scope of the invention, it is intended that all matters contained in the foregoing description or shown in the accompanying drawings shall be interpreted as illustrative and not in a limiting sense.

All references cited in the present application are incorporated in their entirety herein by reference to the extent not inconsistent herewith.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall there between. Now that the invention has been described.

What is claimed is:

1. An ultraviolet light-absorbing particle comprising a biocompatible polymer in combination with a commercially-available UV absorber.

2. The ultraviolet light-absorbing particle according to claim 1 wherein the UV absorber is a commercially-available UV absorber.

3. The ultraviolet light-absorbing particle according to claim 1 wherein the UV absorber is a UV absorber belonging to the family of hydroxyphenyl-s-triazines.

4. The ultraviolet light-absorbing particle according to claim 3 wherein the hydroxyphenyl-s-triazine is bemotrizinol.

5. The ultraviolet light-absorbing particle according to claim 1 wherein the UV absorber is selected from the group consisting of 2-(4,6-Diphenyl-1,3,5-triazin-2-yl)-5-[(hexyl)oxy]-phenol 4-[[4,6-bis[[4-(2-ethylhexoxy-oxomethyl)phenyl]amino]-1,3,5-triazin-2-yl]amino]benzoic acid 2-ethylhexyl ester (ethylhexyl triazone), 2-(2-Hydroxy-4-methoxyphenyl)-4,6-diphenyl-1,3,5-triazine, 2-(4,6-Bis-(2,4-dimethylphenyl)-1,3,5-triazin-2-yl)-5-(octyloxy)-phenol, 2-[4-[2-hydroxy-3-tridecyl oxypropyl]oxy]-2-hydroxyphenyl]-4,6-bis(2,4-dimethylphenyl)-1,3,5-triazine and 2-[4-[2-hydroxy-3-dodecyl oxypropyl]oxy]-2-hydroxyphenyl]-4,6-bis(2,4-dimethylphenyl)-1,3,5-triazine (Tinuvin® 400), 2-[2-Hydroxy-4-[3-(2-ethylhexyl-1-oxy)-2-hydroxypropyloxy]phenyl]-4,6-bis(2,4-dimethylphenyl)-1,3,5-triazine (Tinuvin® 405), 6-[2,6-bis(2,4-dimethylphenyl)-1H-1,3,5-triazin-4-ylidene]-3-(6-methylheptoxy)cyclohexa-2,4-dien-1-one, 2,4-Bis(2-hydroxy-4-butyloxyphenyl)-6-(2,4-bis-butyloxyphenyl-1,3,5-triazine (Tinuvin® 460), Isooctyl 2-[4-[4,6-bis[(1,1′-biphenyl)-4-yl]-1,3,5-triazin-2-yl]-3-hydroxyphenoxy]propanoate (Tinuvin® 479), 2-(2′-hydroxy-5-methylphenyl)-5-benzotriazole, 2-(2H-benzotriazol-2-yl)-4-(1,1,3,3-tetramethylbutyl)phenol, 2-(2′-Hydroxy-3′-t-butyl-5′-methylphenyl)-5-chlorobenzotriazole, 2-(2-hydroxy-3,5-di(1,1-dimethyl-benzyl)-2-benzotriazole, α [3 [3-(2H-Benzotriazol-2-yl)-5-(1,1-dimethylethyl)-4-hydroxyphenyl]-1-oxopropyl]-ω-hydroxypoly(oxo-1,2-ethanediyl), α-[343-(2H-Benzotriazol-2-yl)-5-(1,1-dimethylethyl)-4-hydroxyphenyl]-1-oxopropyl]-ω-[3-[3-(2H-benzotriazol-2-yl)-5-(1,1-dimethylethyl)-4-hydroxyphenyl]-1-oxopropoxy]poly(oxy-1,2-ethanediyl), 2-(2H-Benzotriazol-2-yl)-4,6-bis(1-methyl-1-phenylethyl)phenol (Tinuvin® 900), 2-(2H-Benzotriazol-2-yl)-6-(1-methyl-1-phenylethyl)-4-(1,1,3,3-tetramethylbutyl)phenol (Tinuvin® 928), and combinations thereof.

6. The ultraviolet light-absorbing particle according to claim 1 further comprising a photo-stabilizer to inhibit photodegradation of the UV-absorber, thereby increasing the service life of the UV absorber.

7. The ultraviolet light-absorbing particle according to claim 6 wherein the photostabilizer is a hindered amine.

8. The ultraviolet light-absorbing particle according to claim 7 wherein the hindered amine is 2,2,6,6-tetramethylpiperidine, an alkylated or hydroxylamine analog of 2,2,6,6-tetramethylpiperidine, or a polymer containing any of these functional groups.

9. The ultraviolet light-absorbing particle according to claim 1 wherein the ultraviolet light-absorbing particle is suitable for injection into the dermal layer of the skin and the particle is in the form of (A) Polymer particles, (B) Molecular aggregates, (C) Inorganic nano- or microparticles, (D) Surface-coated nano- or microparticles, (E) Core-shell nano- or microparticles, or (F) Mesoporous nano- or microparticles.

10. The ultraviolet light-absorbing particle according to claim 1 in combination with a tattooable biosensor sensitive to ion concentrations, pH, or glucose levels.

11. The ultraviolet light-absorbing particle according to claim 1 wherein the polymer is a polymer selected from the group consisting of poly(methyl methacrylate) (PMMA), polylactic acid (PLA), poly(lactic-co-glycolic acid) (PLGA), poly(dimethylsiloxane) (PDMS), polyethylene glycol (PEG), Melamine-formaldehyde, Methacrylamide chitosan.

12. The ultraviolet light-absorbing particle according to claim 1 wherein the commercially-available UV absorber is a UV absorber selected from the group consisting of hydroxybenzophenone, hydroxyphenyl-s-triazine, and 2-(2-hydroxyphenyl)benzotriazole, oxalanilide, Aminobenzoic acid, Avobenzone, Cinoxate, Dioxybenzone, Homosalate, Meradimate, Octocrylene, Octinoxate, Octisalate, Oxybenzone, Padimate O, Ensulizole, Sulisobenzone, Titanium dioxide, Trolamine salicylate, and Zinc oxide and derivatives and/or combinations thereof.

13. The ultraviolet light-absorbing particle according to claim 1 further comprising an antioxidant.

14. The ultraviolet light-absorbing particle according to claim 13 wherein the antioxidant is selected from the group consisting of polyphenols, vitamins, carotenoids, hindered phenols, phosphites, melanin or combinations thereof.

15. The ultraviolet light-absorbing particle according to claim 14 wherein the polyphenol is a polyphenol selected from the group consisting of flavonoids, hydroxycinnamic and hydroxybenzoic acids, tannin, cucurmin, gingerol, and combinations thereof.

16. The ultraviolet light-absorbing particle according to claim 14 wherein the vitamin is a vitamin selected from the group consisting of vitamins A, C, E, or combinations thereof.

17. The ultraviolet light-absorbing particle according to claim 14 wherein the carotenoid is a carotenoid selected from the group consisting of beta-carotene, lycopene, or combinations thereof.

18. The ultraviolet light-absorbing particle according to claim 1 wherein the particle is suspended in a biocompatible solvent selected from the group consisting of water, alcohols (e.g., ethanol, isopropanol, glycerol, oligo- and polyethylene glycols), oils (e.g., vegetable oils/triglycerides, geraniol, squalene, etc.), or combinations thereof.

19. The ultraviolet light-absorbing particle according to claim 18 wherein the biocompatible solvent is water, ethanol, isopropanol, glycerol, oligo- and polyethylene glycols, vegetable oils/triglycerides, geraniol, squalene and combinations thereof.

20. The ultraviolet light-absorbing particle according to claim 1 further comprising an additive selected from the group consisting of (i) antiseptics to prevent bacterial contamination, (ii) biocompatible surfactants to stabilize the dispersions and adjust surface tension, (iii) thickening agents to increase viscosity and reduce pigment sedimentation rates (iv) thixotropic agents (e.g. silica) to promote shear thinning (v) preservatives/binding agents (e.g. polyethers, polyvinylpyrrolidinone) to help prevent the inks from drying and to help them bind to needles, (vi) astringents to minimize bleeding in the skin upon implantation, (vii) anesthetics to minimize pain during ink implantation, and combinations thereof.

21. The ultraviolet light-absorbing particle according to claim 20 wherein the antiseptic is an alcohol.

22. The ultraviolet light-absorbing particle according to claim 21 wherein the alcohol is selected from the group consisting of ethanol, isopropanol, glycerol, and poly(ethylene glycol).

23. The ultraviolet light-absorbing particle according to claim 20 wherein the biocompatible surfactant is a polysorbate, TWEEN-20, TWEEN-80 or poly(vinyl alcohol).

24. The ultraviolet light-absorbing particle according to claim 20 wherein the thickening agent is xanthan gum, polyacrylates, polyglycols or combinations thereof.

25. The ultraviolet light-absorbing particle according to claim 24 wherein the polyacrylate is selected from the group consisting of poly(acrylic acid) and co-polymers of poly(acrylic acid).

26. The ultraviolet light-absorbing particle according to claim 24 wherein the polyglycol is selected from the group consisting of poly(ethylene glycol) and poly(propylene glycol).

27. The ultraviolet light-absorbing particle according to claim 20 wherein the thickening agent is methyl acrylate, methyl methacrylate, ethyl acrylate, propyl acrylate, butyl acrylate or combinations thereof.

28. The ultraviolet light-absorbing particle according to claim 1 further comprising TWEEN-80 surfactant at ratio of <1.0% (v/v) to stabilize the suspension, and polyethylene glycol (molecular weight 1000) or glycerol added at a ratio of 10%-30%, whereby the polyethylene glycol or glycerol can act as an antiseptic agent, thickener, or binder.

29. The ultraviolet light-absorbing particle according to claim 1 wherein the particle is in the microparticle to nano-particle size range.

30. An ultraviolet (UV) light-absorbing particle comprising poly(methyl methacrylate) (PMMA) in combination with a UV absorber.

31. The ultraviolet light-absorbing particle according to claim 30 wherein the UV absorber is a commercially-available UV absorber.

32. The ultraviolet light-absorbing particle according to claim 31 wherein the commercially-available UV absorber is selected from the group consisting of 2-hydroxybenzophenone, hydroxyphenyl-s-triazine, and 2-(2-hydroxyphenyl)benzotriazole, oxalanilide, Aminobenzoic acid, Avobenzone, Cinoxate, Dioxybenzone, Homosalate, Meradimate, Octocrylene, Octinoxate, Octisalate, Oxybenzone, Padimate 0, Ensulizole, Sulisobenzone, Titanium dioxide, Trolamine salicylate, Zinc oxide and derivatives and/or combinations thereof.

33. The ultraviolet light-absorbing particle according to claim 30 wherein the ultraviolet (UV) light-absorbing particle is a core-shell particle or nano/microcapsule having a core comprising a UV absorber within a shell or capsule comprising PMMA.

34. The ultraviolet light-absorbing particle according to claim 30 wherein the UV absorber is randomly dispersed in a PMMA matrix.

35. A formulation of transparent or nearly transparent nanoparticles and/or microparticles, wherein the nanoparticles or microparticles are highly absorptive in the UVA and UVB range, in combination with a biocompatible solvent suitable for injection into the dermal or intradermal layer of the skin.

36. The formulation according to claim 35 further comprising an ink or pigment suitable for dermal implantation.

37. A method of implanting an ultraviolet light-absorbing particle into the skin of a subject comprising the steps of:
providing a composition comprising any one of the particles or formulations according to claims 1 through 36;
contacting the skin with a microneedle having the provided composition; and
penetrating the contacted skin with the microneedle.

38. The method of implanting an ultraviolet light-absorbing particle according to claim 37 wherein the microneedle is a dissolving microneedle.

39. The method of implanting an ultraviolet light-absorbing particle according claim 38 wherein the dissolving microneedle comprises a suitable carrier selected from the group consisting of polyvinylpyrrolidinone or polyvinyl alcohol and their liquid pre-polymers, or aqueous solutions of carboxymethyl cellulose, trehalose, maltodextrin, galactose, glucose, and silk.

40. A method of implanting an ultraviolet light-absorbing particle comprising the steps of:
providing a composition comprising any one of the particles or formulations according to claims 1 through 36;
contacting the skin with a needle-free tattoo machines configured to deliver the provided composition in combination with a tattoo ink; and
penetrating the contacted skin with the composition in combination with tattoo ink droplets at sufficiently high velocity to penetrate into the dermis.

41. A method of implanting an ultraviolet light-absorbing particle comprising the steps of:
providing a composition comprising any one of the particles or formulations according to claims 1 through 36;
contacting the skin with an (electric) tattoo machine (rotary or coil) configured to deliver the provided composition in combination with a tattoo ink; and
penetrating the contacted skin with the composition in combination with tattoo ink droplets under conditions sufficient to penetrate into the dermis.

42. The ultraviolet light-absorbing particle according to any one of claims 1-36 in combination with a tattooable UV sensor.
